(19) United States
(12) Patent Application Publication
(10) Pub. No.: US 2012/0177910 A1
(43) Pub. Date: Jul. 12, 2012

(54) Coated Medical Devices

(75) Inventors: Jan Weber, Maastricht (NL); Aiden Flanagan, Co. Galway (IE)

(73) Assignee: BOSTON SCIENTIFIC SCIMED, INC., Maple Grove, MN (US)

(21) Appl. No.: 13/347,441

(22) Filed: Jan. 10, 2012

Related U.S. Application Data
(60) Provisional application No. 61/431,740, filed on Jan. 11, 2011.

(51) Int. Cl. B32B 5/16; A61L 33/00; B82Y 99/00

(52) U.S. Cl. 428/323; 427/2.25; 977/773

(57) ABSTRACT

The present invention relates to medical devices, and in particular, medical devices that have an inorganic coating, e.g., a coating capable of releasing inorganic nanoparticles into a passageway to be treated with the medical device.

### CROSS-REFERENCE TO RELATED APPLICATION

This application claims priority under 35 USC §119(e) to U.S. Provisional Patent Application Ser. No. 61/431,740, filed on Jan. 11, 2011, the entire contents of which are hereby incorporated by reference.

### TECHNICAL FIELD

The present invention relates to medical devices, and in particular, medical devices that have an inorganic coating.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721, the entire contents of which is hereby incorporated by reference herein. The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

### SUMMARY

Inorganic nanoparticles can be administered to patients to exert therapeutic effects, e.g., blockage of inflammatory responses, inhibition of oxidative stress, reduction of cell proliferation, among others. The present invention is based, at least in part, on the discovery that a layer(s) can be deposited on implantable devices (e.g., stents, pacing leads, balloons, vascular closing devices, etc.), which can release a flux of appropriately-sized nanoparticles to a patient after implantation in the body.

Accordingly, in one aspect, the disclosure features a medical device that includes a surface; a plurality of inorganic nanoparticles disposed on at least a portion of the surface; and a degradable inorganic adhesion layer, wherein the layer is disposed over the plurality of inorganic nanoparticles and adheres the nanoparticles to the surface. The medical device can be any medical device, e.g., an implantable device, e.g., a stent, pacing lead, vascular closing device, or balloon.

Any plurality of inorganic nanoparticles described herein can include metal oxide nanoparticles. An inorganic nanoparticle can include, e.g., cerium, yttrium, titanium, iron, aluminum, tantalum, or gold, or a mixture of at least two thereof. In some instances, all inorganic nanoparticles in a plurality can be composed of the same inorganic material. Alternatively, a plurality of nanoparticles can include at least two groups of nanoparticles, wherein each group of nanoparticles is composed of a material that is different from all other groups in the plurality.

In any plurality of inorganic nanoparticles described herein, the nanoparticles in the plurality can be of relatively equal size. In some instances, the plurality of nanoparticles can include at least two groups of nanoparticles, wherein each group of nanoparticles is of a different size from all other groups in the plurality. A plurality of nanoparticles can include nanoparticles that are, e.g., of a size ranging from about 5 to about 30 nm, e.g., about 10 to about 20 nm.

Any plurality of nanoparticles described herein can include hybrid nanoparticles. Hybrid nanoparticles include more than one material, e.g., more than one metal oxide (e.g., two, three, four, or more, metal oxides). In some instances, hybrid nanoparticles can include a core comprising iron oxide and an outer coating comprising cerium oxide.

In some instances, a plurality of nanoparticles described herein can include ultrasmall superparamagnetic particles of iron oxide (USPIOs) and cerium oxide nanoparticles. The USPIOs and cerium oxide nanoparticles can be, e.g., in the range of about 18 to about 30 nm. The ratio of USPIOs to cerium oxide nanoparticles in the plurality can be, e.g., in a range of about 1:1 to about 1:10, e.g., about 1:2, about 1:5 or about 1:8.

Any degradable inorganic adhesion layer described herein can include, e.g., aluminum oxide, silicon dioxide, zinc oxide, or any combination thereof. The degradable inorganic adhesion layer can be, e.g., of a thickness in the range of about 1 angstrom to about 1 micrometer. For example, the degradable inorganic adhesion layer can be of a thickness in the range of about 1 nanometer to about 20 nanometers, e.g., of a thickness of about 2 nanometers.

Where a medical device described herein includes an inorganic adhesion layer, the medical device can in some instances further include a therapeutic agent disposed on or over the inorganic adhesion layer. For example, the therapeutic agent can be everolimus. In some instances, the degradable inorganic adhesion layer disposed over the plurality of inorganic nanoparticles is a first adhesion layer, and the medical device further comprises a second degradable inorganic adhesion layer disposed over the therapeutic agent.

In one embodiment, the disclosure provides a medical device that includes a surface; a plurality of cerium oxide nanoparticles disposed on at least a portion of the surface; and a degradable inorganic adhesion layer, wherein the layer is disposed over the plurality of cerium oxide nanoparticles and adheres the nanoparticles to the surface.

In another aspect, the disclosure provides a medical device that includes a surface and a plurality of aggregated particles disposed on the surface, wherein each aggregated particle comprises a carrier agent particle portion (e.g., a therapeutic agent particle portion) and an inorganic nanoparticle portion. The medical device can be, e.g., an implantable device, e.g., a stent, pacing lead, vascular closing device, or balloon. In some instances, the surface, or portion thereof, of the medical device can be roughened and include a plurality of invaginations. At least a portion of the plurality of aggregated nanoparticles can be disposed within the invaginations. The carrier agent can include a therapeutic agent, e.g., paclitaxel. The plurality can comprise aggregated nanoparticles wherein the inorganic nanoparticle portions comprise cerium, yttrium, titanium, iron, aluminum, tantalum, or gold, or a mixture of at least two thereof. The inorganic nanoparticle portions can be, e.g., hybrid nanoparticle portions, such as hybrid nanoparticle portions that include a core comprising iron oxide and an outer coating comprising cerium oxide.

Any plurality of aggregated nanoparticles described herein can include at least two groups of aggregated nanoparticles, wherein the carrier agent portions of the aggregated nanoparticles in each group comprise a carrier agent that is different from all other groups in the plurality. Likewise, any plurality of aggregated nanoparticles described herein can include at least two groups of aggregated nanoparticles, wherein the inorganic nanoparticle portions of the aggregated nanoparticles in each group comprise an inorganic agent that is different from all other groups in the plurality. In some instances, the size ratio of a carrier agent nanoparticle to inorganic nanoparticle in at least a portion of a plurality of aggregated nanoparticles is in a range of about 2:1 to about 60:1, e.g., about 20:1 to about 60:1, e.g., about 20:1. The carrier agent nanoparticle portion in at least a portion of a plurality of aggregated nanoparticles can be, e.g., about 50 to about 300 nm. The inorganic nanoparticle portion in at least a portion of a plurality of aggregated nanoparticles can be, e.g., about 5 to 40 nm in diameter. In some instances, a plurality can include aggregated nanoparticles wherein the inorganic nanoparticle portion is encapsulated within the carrier agent portion.

Any plurality of aggregated nanoparticles described herein can include aggregated nanoparticles wherein the inorganic nanoparticle portions include ultrasmall superparamagnetic particles of iron oxide (USPIOs) and cerium oxide nanoparticles. The USPIOs and cerium oxide nanoparticles can be, e.g., in the range of about 18 to about 30 nm. The ratio of USPIOs to cerium oxide nanoparticles in the plurality can be in a range of about 1:1 to about 1:10, e.g., about 1:2, about 1:5, or about 1:8.

In another embodiment, the disclosure provides a medical device that includes a surface and a plurality of aggregated nanoparticles disposed on the surface, wherein the aggregated nanoparticles comprise a therapeutic agent nanoparticle portion and a cerium oxide nanoparticle portion. The therapeutic agent nanoparticle portion can include, e.g., paclitaxel.

In yet another aspect, the disclosure provides a medical device that includes a surface having a plurality of cerium oxide nanoparticles disposed thereon.

In still another aspect, the disclosure provides a medical device that includes a surface having a plurality of hybrid nanoparticles disposed thereon. The hybrid nanoparticles can have, for example, a core that includes iron oxide and an outer coating that includes an oxide, e.g., cerium oxide.

Any medical device described herein can further include a module, e.g., a magnet or other element or set of elements, capable of generating a magnetic field. The magnetic field can be used to retain nanoparticles, e.g., hybrid nanoparticles, to the surface of the medical device. The module can be controllable, e.g., by an outside user, such that the intensity of the magnetic field can be increased and/or decreased (e.g., such that the magnetic field can be turned on and off), and the nanoparticles can be retained to or released from the surface.

In still another aspect, the disclosure provides a method of making a medical device, which includes providing a substrate comprising a surface; depositing a plurality of inorganic nanoparticles on at least a portion of the surface, wherein the nanoparticles are in loose communication with the surface; and depositing a degradable inorganic adhesion layer over the plurality of inorganic nanoparticles, wherein the adhesion layer adheres the nanoparticles to the surface. The inorganic nanoparticles can be deposited, e.g., using nanocluster deposition, e.g., at a voltage of about 50 to about 500 Volts (e.g., at a voltage of about 500 Volts. The degradable adhesion layer can be deposited using atomic layer deposition (ALD). In some instances, the degradable adhesion layer can be deposited to a thickness of about 1 nm to about 5 nm. In other instances, the method can further include depositing a therapeutic agent on the degradable adhesion layer. In some other instances, the degradable inorganic adhesion layer disposed over the plurality of inorganic nanoparticles is a first adhesion layer, and the method further comprises depositing a second degradable inorganic adhesion layer over the therapeutic agent.

In yet another aspect, the disclosure provides a method of making a medical device, which includes providing a substrate comprising a surface; depositing a plurality of carrier nanoparticles on the surface; and accelerating a plurality of inorganic nanoparticles toward the plurality of carrier agent nanoparticles, to thereby aggregate at least a portion of the plurality of inorganic nanoparticles to at least a portion of the plurality of carrier agent nanoparticles and provide a plurality of aggregated nanoparticles.

In still another aspect, the disclosure provides a method of making a medical device, which includes providing a substrate comprising a surface; providing a plurality of aggregated nanoparticles, wherein the aggregated nanoparticles comprise a carrier agent nanoparticle portion and an inorganic nanoparticle portion; and depositing the plurality of aggregated nanoparticles on the surface. The plurality of aggregated nanoparticles can be provided by accelerating a plurality of inorganic nanoparticles toward a plurality of carrier agent nanoparticles, to thereby aggregate at least a portion of the plurality of inorganic nanoparticles to at least a portion of the plurality of carrier agent nanoparticles.

In any methods described herein, a plurality of inorganic nanoparticles can be accelerated toward a plurality of carrier agent nanoparticles by nanocluster deposition. The size ratio of carrier agent nanoparticles (e.g., therapeutic agent nanoparticles) to inorganic nanoparticle in at least a portion of a plurality of aggregated nanoparticles described herein can be, e.g., in a range of about 2:1 to about 60:1, e.g., about 20:1 to 60:1, e.g., about 20:1. A carrier agent nanoparticle portion in at least a portion of a plurality can be, e.g., about 50-300 nm. An inorganic nanoparticle portion can be, e.g., about 5 to 40 nm.

In any methods described herein, a surface of a medical device can be roughened, to thereby provide a surface comprising a plurality of invaginations. Inorganic nanoparticles, hybrid nanoparticles, and/or aggregated nanoparticles can be deposited in the invaginations.

In yet another aspect, the disclosure provides a method of making a medical device, which includes providing a device comprising a surface; and depositing a plurality of cerium oxide nanoparticles on at least a portion of the surface.

In another aspect, the disclosure provides a method of making a medical device, comprising: providing a substrate comprising a surface; providing a plurality of hybrid nanoparticles, e.g., wherein the hybrid nanoparticles comprise a core that comprises iron oxide and an outer coating the comprises cerium oxide; and depositing the plurality of hybrid nanoparticles on the surface.

In still another aspect, the disclosure provides a method of treating a vessel in a patient, comprising: inserting and actuating a medical device described herein in a vessel in the patient to thereby treat the vessel; and imaging, e.g., using magnetic resonance imaging, the vessel in the patient to thereby observe treatment of the vessel.

In yet another aspect, the disclosure provides a method of treating a vessel in a patient, comprising: inserting into the patient's vessel a medical device that comprises: (i) a surface having a plurality of nanoparticles (e.g., hybrid nanoparticles) disposed thereon, e.g., wherein the plurality comprises hybrid nanoparticles having a core that comprises iron oxide and an outer coating comprising cerium oxide; and (ii) a module capable of generating a magnetic field to thereby retain the nanoparticles on the surface, and wherein the magnetic field is activated when the medical device is inserted into the patient's vessel; and reducing the intensity of the magnetic field, to thereby release at least a portion of the nanoparticles from the surface.

In one aspect, the disclosure provides a medical device, comprisinga surface; a plurality of inorganic nanoparticles disposed on at least a portion of the surface; and a degradable inorganic adhesion layer, wherein the layer is disposed over the plurality of inorganic nanoparticles and adheres the nanoparticles to the surface. The medical device can be an implantable device. The plurality of inorganic nanoparticles can include metal oxide nanoparticles. The plurality of nanoparticles can include cerium, yttrium, titanium, iron, aluminum, tantalum, or gold nanoparticles, or a mixture of any two or more thereof. The degradable inorganic adhesion layer can include aluminum oxide, silicon dioxide, or zinc oxide. The plurality of nanoparticles can include ultrasmall superparamagnetic particles of iron oxide (USPIOs) and cerium oxide nanoparticles. The plurality of nanoparticles can include hybrid nanoparticles that include an iron oxide core and an outer cerium oxide coating. The degradable inorganic adhesion layer can be of a thickness in the range of about 1 angstrom to about 1 micrometer. The medical device can include a therapeutic agent disposed on the inorganic adhesion layer.

In another aspect, the disclosure provides a medical device, comprising a surface; and a plurality of aggregated particles disposed on the surface, wherein each aggregated particle comprises a carrier agent particle portion and an inorganic nanoparticle portion. The medical device can be an implantable device. The surface can be roughened and include a plurality of invaginations. At least a portion of the plurality of aggregated nanoparticles can be disposed within the invaginations. The plurality can include aggregated nanoparticles wherein the carrier agent particle portion comprises paclitaxel. The plurality can include aggregated nanoparticles wherein the inorganic nanoparticle portions comprise cerium, yttrium, titanium, iron, aluminum, tantalum, or gold, or a mixture of at least two thereof. The plurality can include aggregated nanoparticles wherein the inorganic nanoparticle portions include ultrasmall superparamagnetic particles of iron oxide (USPIOs) and cerium oxide nanoparticles. The plurality can include aggregated nanoparticles wherein the inorganic nanoparticle portions are hybrid nanoparticles comprising an iron oxide core and an outer oxide coating. The size ratio of carrier agent nanoparticle to inorganic nanoparticle in at least a portion of the plurality of aggregated nanoparticles can be in a range of about 2:1 to about 60:1.

In yet another aspect, the disclosure provides a medical device, comprising: a surface having a plurality of nanoparticles disposed thereon, wherein the plurality comprises hybrid nanoparticles having a core that comprises iron oxide and an outer coating comprising cerium oxide; and a module capable of generating a magnetic field to thereby retain the hybrid nanoparticles on the surface.

In one aspect, the disclosure provides a method of making a medical device, comprising: providing a substrate comprising a surface; depositing a plurality of inorganic nanoparticles on at least a portion of the surface, wherein the nanoparticles are in loose communication with the surface; and depositing a degradable inorganic adhesion layer over the plurality of inorganic nanoparticles, wherein the adhesion layer adheres the nanoparticles to the surface. The inorganic nanoparticles can be deposited using nanocluster deposition. The degradable adhesion layer can be deposited using atomic layer deposition. The method can further include depositing a therapeutic agent on the degradable adhesion layer.

In another aspect, the disclosure provides a method of making a medical device, comprising: providing a substrate comprising a surface; depositing a plurality of carrier nanoparticles on the surface; and accelerating a plurality of inorganic nanoparticles toward the plurality of carrier agent nanoparticles, to thereby aggregate at least a portion of the plurality of inorganic nanoparticles to at least a portion of the plurality of carrier agent nanoparticles and provide a plurality of aggregated nanoparticles. The plurality of inorganic nanoparticles can be accelerated toward the plurality of carrier agent nanoparticles by nanocluster deposition. In the method, providing a device comprising a surface can include roughening the surface, to thereby provide a surface comprising a plurality of invaginations.

In still another aspect, the disclosure provides a method of making a medical device, comprising: providing a substrate comprising a surface; providing a plurality of aggregated nanoparticles, wherein the aggregated nanoparticles comprise a carrier agent nanoparticle portion and an inorganic nanoparticle portion; and depositing the plurality of aggregated nanoparticles on the surface. The plurality of aggregated nanoparticles can be provided by accelerating a plurality of inorganic nanoparticles toward a plurality of carrier agent nanoparticles, to thereby aggregate at least a portion of the plurality of inorganic nanoparticles to at least a portion of the plurality of carrier agent nanoparticles. The plurality of inorganic nanoparticles can be accelerated toward the plurality of carrier agent nanoparticles by nanocluster deposition. In the method, providing a device comprising a surface can include roughening the surface, to thereby provide a surface comprising a plurality of invaginations.

In yet another aspect, the disclosure provides a method of making a medical device, comprising: providing a substrate comprising a surface; providing a plurality of hybrid nanoparticles, wherein the hybrid nanoparticles comprise a core that comprises iron oxide and an outer coating that comprises cerium oxide; and depositing the plurality of hybrid nanoparticles on the surface.

In one aspect, the disclosure provides a method of treating a vessel in a patient, comprising: inserting and actuating in the vessel a medical device comprising a surface; a plurality of inorganic nanoparticles disposed on at least a portion of the surface; and a degradable inorganic adhesion layer, wherein the layer is disposed over the plurality of inorganic nanoparticles and adheres the nanoparticles to the surface, and wherein the plurality of nanoparticles includes ultrasmall superparamagnetic particles of iron oxide (USPIOs) and cerium oxide nanoparticles, to thereby treat the vessel; and imaging the vessel in the patient to thereby observe treatment of the vessel in the patient. Imaging can be performed using magnetic resonance imaging (MRI).

In another aspect, the disclosure provides a method of treating a vessel in a patient, comprising: inserting and actuating in the vessel a medical device comprising a surface; a plurality of aggregated particles disposed on the surface; wherein each aggregated particle comprises a carrier agent particle portion and an inorganic nanoparticle portion, and wherein the plurality includes aggregated nanoparticles wherein the inorganic nanoparticle portions include ultrasmall superparamagnetic particles of iron oxide (USPIOs) and cerium oxide nanoparticles, to thereby treat the vessel; and imaging the vessel in the patient to thereby observe treatment of the vessel in the patient. Imaging can be performed using magnetic resonance imaging (MRI).

In still another aspect, the disclosure provides a method of treating a vessel in a patient, comprising: inserting into the patient's vessel a medical device that comprises: (i) a surface having a plurality of nanoparticles disposed thereon, wherein the plurality comprises hybrid nanoparticles having a core that comprises iron oxide and an outer coating comprising cerium oxide; and (ii) a module capable of generating a magnetic field to thereby retain the hybrid nanoparticles on the surface, and wherein the magnetic field is activated when the medical device is inserted into the patient's vessel; and reducing the intensity of the magnetic field, to thereby release at least a portion of the hybrid nanoparticles from the surface.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-1C are longitudinal cross-sectional views illustrating delivery of a stent in a collapsed state, expansion of the stent, and deployment of the stent.

FIG. 2 is a perspective view of a stent.

FIGS. 3A-3D are cross sectional views of medical device surfaces that include a plurality of inorganic nanoparticles coated with a degradable inorganic adhesion layer.

FIG. 4 is a cross-sectional view of a medical device surface that includes a plurality of aggregated particles, which include a therapeutic agent particle portion and an inorganic nanoparticle portion.

FIG. 5 is a picture of a roughened balloon surface.

FIGS. 6A-6B are pictures of a stent made in accordance with the methods described herein.

FIG. 6C is a graph illustrating the results of drug-release tests from stents made in accordance with the methods described herein.

FIG. 7 is picture providing a close-up view of a stent made in accordance with the methods described herein.

Like reference symbols in the various drawings indicate like elements.

### DETAILED DESCRIPTION

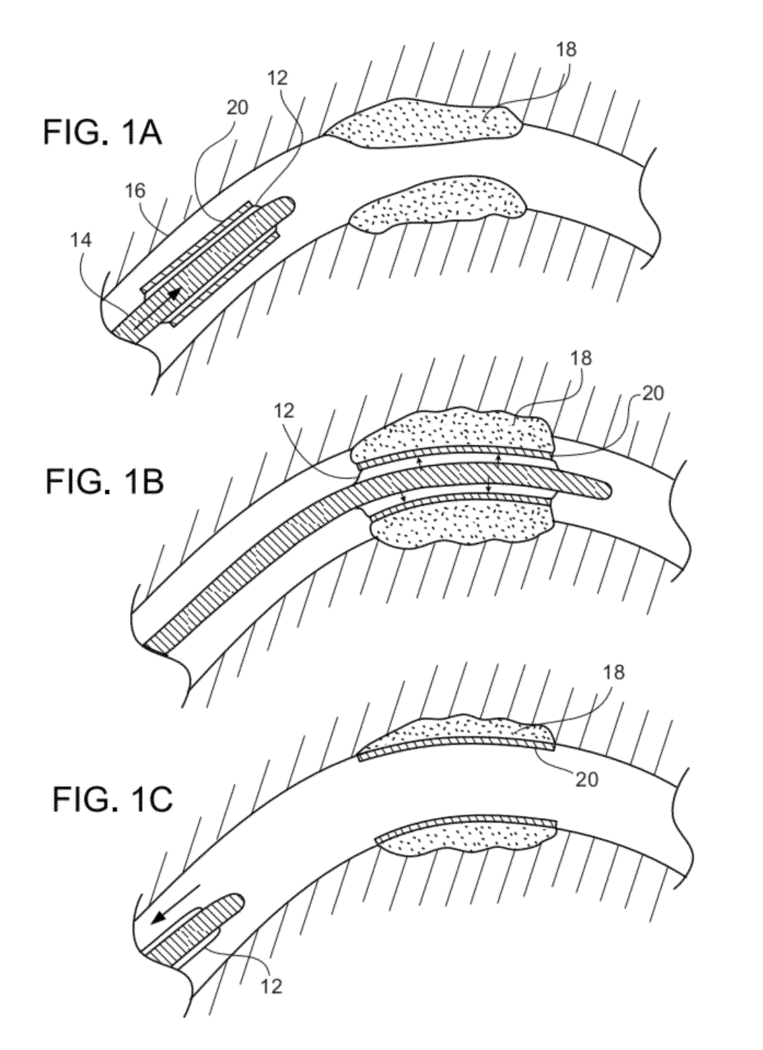

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

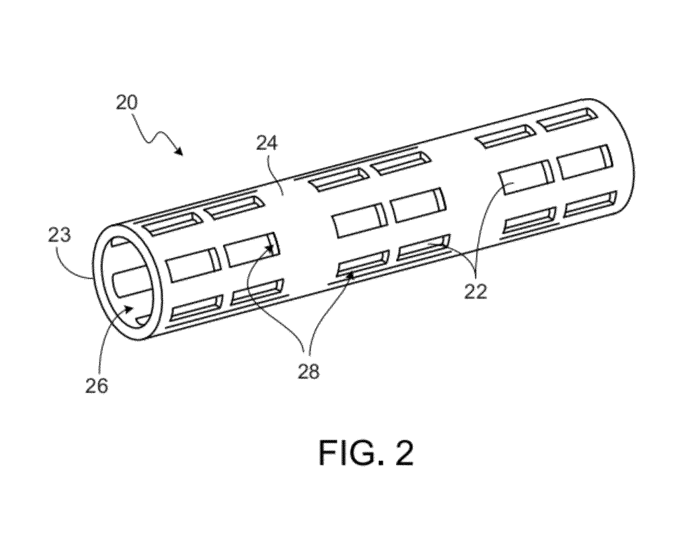

Referring to FIG. 2, an example of one stent 20 includes a plurality of fenestrations 22 defined in a wall 23. Stent 20 includes several surface regions, including an outer, or abluminal, surface 24, an inner, adluminal, surface 26, and a plurality of cutface surfaces 28. The stent can be balloon expandable, as illustrated above, or a self-expanding stent.

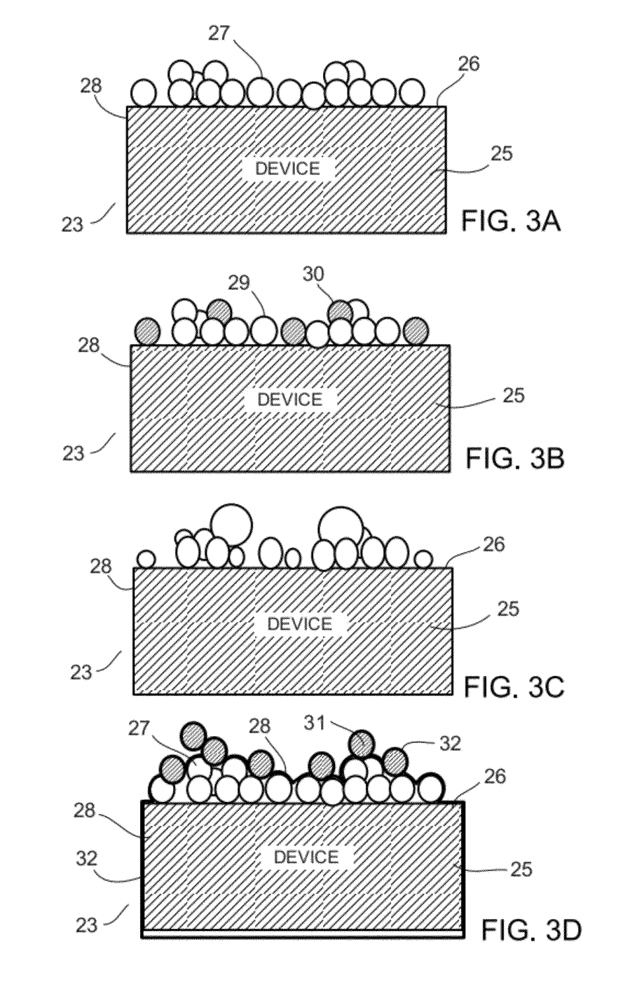

FIGS. 3A to 3D, cross-sectional views, illustrate several embodiments of one aspect of the invention. Referring to FIG. 3A, a stent wall 23 includes a stent body 25 formed, e.g., of metal, and includes a surface 26 upon which a plurality of inorganic nanoparticles 27 are disposed. The inorganic nanoparticles 27 are soft-landed on the surface 26, and at least some members of the plurality may or may not be fused together. In certain embodiments, the plurality of inorganic particles 27 is deposited on the surface 26 as a simple dust layer. Inorganic nanoparticles 27 can be, e.g., about 5 to about 100 nm, e.g., about 5 to about 30 nm, or about 10 to about 20 nm, in diameter. A degradable, inorganic adhesion layer 28 is disposed over the plurality of inorganic nanoparticles 27. Where an adhesion layer is described as being “disposed over” another element, at least a portion of the adhesion layer is disposed further away from the surface of the medical device than the other element. As such, in this case, the degradable adhesion layer being disposed over the nanoparticles includes the adhesion layer covering the soft-landed inorganic nanoparticles in a conformal coating that acts, e.g., as a degradable inorganic “glue.” Inorganic adhesion layer 28 adheres the inorganic nanoparticles 27 to each other and/or to surface 26. Adhesion layer 28 can be, e.g., of a thickness of about 10 to about 30 nm, e.g., about 15 to about 25 nm or about 16 to 20 nm. FIG. 3B illustrates a configuration wherein the plurality of inorganic nanoparticles 27 comprises at least two groups, wherein each group is composed of a material, e.g., cerium oxide 29 and aluminum oxide 30, that is different from any other group. FIG. 3C illustrates another configuration wherein the plurality of inorganic nanoparticles 27 includes nanoparticles of various sizes. FIG. 3D illustrates a configuration wherein a second plurality of inorganic nanoparticles 31 is deposited on adhesion layer 28, which is disposed over a first layer of inorganic nanoparticles 27, and wherein a second inorganic adhesion layer 32 is deposited over the second plurality of inorganic nanoparticles 31. Alternatively or in addition, a contiguous or non-continguous layer of a therapeutic agent, such as everolimus, can be disposed over a degradable inorganic adhesion layer described herein.

Skilled practitioners will appreciate that an inorganic adhesion layer can be of any suitable thickness, and the choice of thickness can depend on a number of variables, e.g., the material used, the length of the period over which the inorganic nanoparticles are to be released in the passageway, the dosage of the nanoparticles desired, the type of passageway to be treated, and the device on which the layer is to be disposed. The thickness can, therefore, range from 1 angstrom to one micrometer, e.g., from about 1 nm to about 50 nm, e.g., about 10 nm to about 20 nm.

Such configurations of nanoparticles and inorganic adhesion layers allow controlled release of the inorganic nanoparticles, e.g., precisely sized inorganic nanoparticles. That is, a practitioner may position a device described herein temporarily or permanently against tissue within a passageway (e.g., a vessel). The nanoparticles would be released into and/or onto the tissue over time as the adhesion layer degrades within the passageway and loses its ability to adhere the inorganic nanoparticles to the surface.

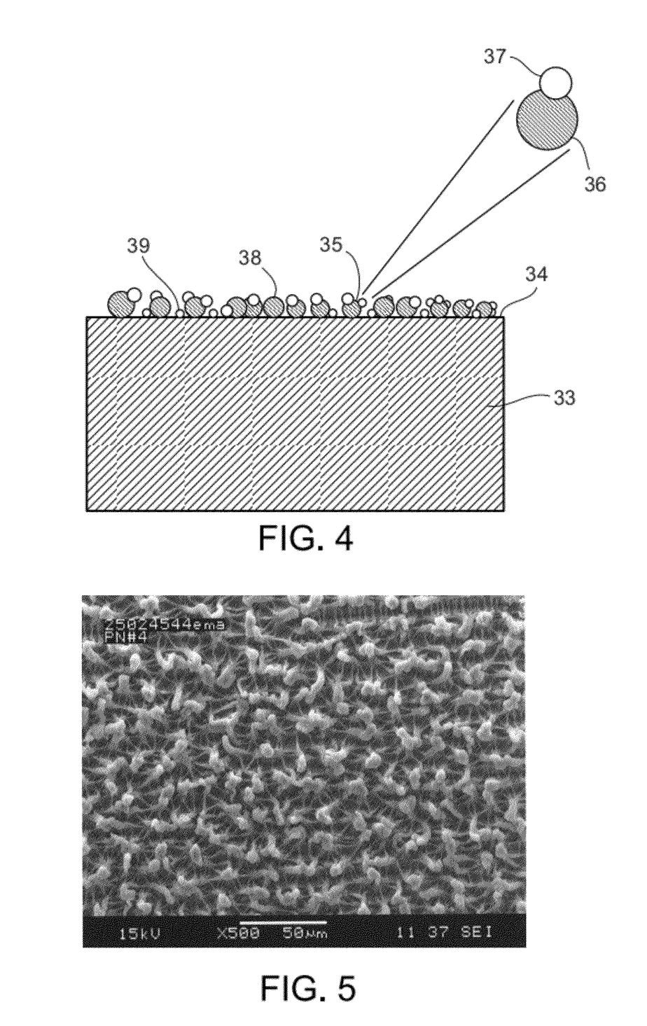

FIG. 4, another cross-sectional view, illustrates another aspect of the invention. Referring to FIG. 4, substrate 33, e.g., a medical device body, comprises a surface 34 upon which a plurality of aggregated particles 35 are disposed. Aggregated particles can include at least a carrier agent particle portion 36 and an inorganic nanoparticle portion 37. In some instances, inorganic particles 39 and carrier agent particles 38, both in non-aggregated form, can be deposited on surface 34 in addition to aggregated particles 35. A carrier agent particle can be comprised of, e.g., a therapeutic agent, an agent that may not itself exert a therapeutic effect (e.g., fatty acids, sugars (e.g., dextran or glucose), citrate, cholesterol, DNA or RNA), or a mixture or combination thereof. Such configurations provide a system wherein an inorganic nanoparticle can be carried by the carrier agent particle into and/or between cells of a tissue to be treated, e.g., the tissue of a vessel wall. As the carrier agent is degraded, the inorganic nanoparticle is released from the aggregated particle, and the inorganic nanoparticle can exert its own therapeutic effect, e.g., by blocking inflammatory responses, inhibiting oxidative stress, reducing cell proliferation, etc. As used herein, an “aggregated particle” is a particle that includes at least two portions, i.e., an inorganic nanoparticle portion and a carrier agent particle portion (e.g., a therapeutic agent particle portion), and wherein the inorganic nanoparticle is physically embedded at least partially, e.g., completely, within the carrier agent particle, such that the two portions remain in communication in suspension.

In some embodiments, the size ratio of carrier particle portion to inorganic nanoparticle portion is about 2.5:1 to about 60:1, e.g., about 5:1 to about 50:1, about 10:1 to about 40:1, or about 20:1 to about 30:1. For example, in some instances, the size ratio is about 20:1, wherein the carrier agent particle is about 100 nm and the inorganic nanoparticle is about 5 nm (e.g., as both are measured prior to aggregation).

In still another aspect, inorganic nanoparticles can be joined, e.g., ionically or covalently, with a carrier agent, e.g., without embedding the inorganic nanoparticle in the carrier agent. The carrier agent can be, e.g., a therapeutic agent, an agent that may not itself exert a therapeutic effect (e.g., fatty acid, sugars (e.g., dextran or glucose), citrate, cholesterol, DNA fragments, RNA fragments), or any mixture or combination thereof. Joining of inorganic nanoparticles to one type, or multiple types, of carrier agents is contemplated. Skilled practitioners will appreciate that inorganic nanoparticles can be joined with carrier agent(s) using any method known in the art, and the choice of such methods will depend, e.g., on the materials to be joined. Diverse functional groups on organic molecules are suitable for use in attachment to metal oxides. For example, groups such as amine, alcohol, ether or thiol groups are useful. One useful example is thiol bonding to gold nano-particles.

The medical device can be any implantable device, e.g., a stent or a balloon. In one embodiment, the surface can be roughened to provide a plurality of invaginations in the surface, in which at least some of the aggregated particles can be disposed. FIG. 5 illustrates the surface of a balloon roughened to provide such invaginations. The surface of a stent can be similarly roughened, e.g., as described in U.S. Ser. No. 12/205,004, (see, e.g., pages 2 and 3, para. [0025] and [0026] of U.S. Patent Publication No. US20100063584), which is incorporated herein by reference.

Inorganic adhesion layers and inorganic nanoparticles can include, or be composed entirely of, a metal oxide. Suitable metal oxides include but are not limited to, metal oxides that contain one or more of the following metals: titanium, scandium, iron, tantalum, cobalt, chromium, manganese, platinum, iridium, niobium, vanadium, zirconium, tungsten, rhodium, ruthenium, gold, copper, zinc, yttrium, molybdenum, technetium, palladium, cadmium, hafnium, rhenium and combinations thereof. Examples of suitable metal oxides include without limitation: cerium oxides, platinum oxides, yttrium oxides, tantalum oxides, titanium oxides, zinc oxides, iron oxides, magnesium oxides, aluminum oxides, iridium oxides, niobium oxides, zirconium oxides, tungsten oxides, rhodium oxides, ruthenium oxides, alumina, zirconia, silicone oxides such as silica based glasses and silicon dioxide, or combinations thereof. Exemplary metal oxides that are particularly useful for biodegradable inorganic adhesion layers include silicon oxide, aluminum oxide and zinc oxide. Use of combinations of metal oxides, e.g., combinations of any of those described herein, are also contemplated.

Of particular use are metal oxides and combinations that facilitate imaging of a patient's treated area, e.g., vessels. For example, ultrasmall superparamagnetic particles of iron oxide (USPIOs), e.g., in the range of about 18 to about 30 nm, are useful. USPIOs in the range of 18-30 nm have been shown to enable one to pinpoint and image, e.g., by magnetic resonance imaging (MRI), human atherosclerosis in vivo (see, e.g., Lipinski et al., Nature Reviews Cardiology 1, 48-55 (1 Nov. 2004), incorporated herein by reference). USPIOs can be used alone or in combination with other metal oxides. For example, one combination is a mixture of USPIOs, e.g., in the range of about 18 to about 30 nm, and cerium oxide nanoparticles, e.g., also in the range of about 18 to about 30 nm. The ratio of USPIOs to cerium oxide nanoparticles can, for example, range from about 1:1 to about 1:10, e.g., about 1:2, 1:5, 1:8, or 1:10. Such a combination would allow both visualization and therapeutic action. Either one or both metal oxides can be bound to a carrier, e.g., as discussed above.

Also of particular use are hybrid nanoparticles, i.e., nanoparticles that each include more than one types of material, e.g., at least 2, 3, 4, or more, types of materials. Skilled practitioners will appreciate that hybrid nanoparticles can be used in any device, layer, and/or aggregated nanoparticle, described herein. Hybrid nanoparticles can be made, e.g., using the Mantis system as described herein, employing a special three sputter target configuration. In this three-sputter configuration, two or more targets are simultaneously sputtered, creating a gas composition of all sputtered elements, agglomerating into nano-particles consisting out of all initial sputtered elements. One useful alternative is to feed mono nano-particles though a second sputter chamber where a second or third target is being sputtered, creating an additional layer of this secondary material onto the initial mono-material. Many different combinations of materials are possible. For example, hybrid nanoparticles can include, e.g., a core that includes, or is composed of, iron oxide, and an outer coating that comprises or is composed of cerium oxide. Such hybrid nanoparticles would allow release of cerium oxide to the patient while also allowing a practitioner to monitor, e.g., using imaging techniques (such as MRI), the location and residence time of the hybrid nanoparticles within a patient's body. Such hybrid nanoparticles would allow practitioners to employ magnetic fields to, e.g., retain the hybrid nanoparticles on a surface of a medical device, such as the surface of a balloon or stent. The hybrid nanoparticles are releasable from the surface when the magnetic field is reduced or terminated. Alternatively or in addition, the hybrid nanoparticles can be held to the surface of a tissue after release from the surface of a medical device, e.g., using a magnet nearby the tissue. Accordingly, also contemplated by the present disclosure are methods of treating a passageway (e.g., a vessel) in a patient using the hybrid nanoparticles and devices as described herein. Further contemplated by the present disclosure are medical devices described herein that include a magnetic field source, which can be used to adhere hybrid nanoparticles to a surface of the medical device. For example, a stent or a balloon can include a module that provides a constant magnetic field, or that can be activated to provide a magnetic field. For example, a medical device described herein can be equipped with a magnet. An exemplary system may include a magnetic wire made out of Neodynium being positioned on the inside of a balloon, for example a ND005110 Neodymium Wire, as can be obtained by Goodfellow Corp. USPIO particles can be positioned within the folds of the balloon (i.e., while the balloon is in a folded configuration) and held at least in part to the balloon system by way of the magnetic force exerted by the magnetic wire. Upon opening of the balloon, the particles would be exposed and the attraction to the magnetic wire would be reduced by the increased distance between the wire at the center of the balloon and the particles disposed on the exterior of the balloon surface.

It will be appreciated that any of the devices and/or coatings described in the present specification can be substantially polymer-free, e.g., completely polymer-free.

The present invention includes methods of making the devices described herein. When depositing particles described herein onto the surface of a medical device, any of the various known methods by which fine particulate materials are deposited onto a substrate can be used. Particularly useful is nanocluster deposition, e.g., the method described in WO/2007034167 (Mantis Deposition, Ltd.), which can be adapted to deposit particles of the present invention onto a medical device, e.g., by electrostatic acceleration of nanoparticles onto the surface of the device. Nanocluster deposition is a technique known to those of skill in the art, and the equipment necessary for carrying out nanocluster deposition is commercially available. In order to lay down a plurality of soft-landed nanoparticles on a surface in accordance with one aspect of the present invention, for example, one might do so at a voltage of about 500 Volt, which would land the particles on the surface and barely fuse the particles together. Lowering the deposition voltage to about 100 Volt or even lower would deposit the nanoparticles without fusing, thereby providing the simple dust layer mentioned above. Particle sizing for deposition can be determined accurately using a quadrupole filter before deposition of the particles on the target. An exemplary quadrupole filter useful in the methods described herein is a MesoQ quadrupole filter, produced by Mantis Deposition, Ltd.

The deposition in the Mantis system is based on creating an electric field between the plasma chamber outlet where the particles are being produced and the target, for example, a grounded stent. However, non-conductive substrates can be coated as well by placing them within the electric field lines between inlet and bias point. This causes the particles to be impinged on the material. An advantage of a non-conductive substrate is that the particles can be deposited under an angle, whereas with conductive substrates, field lines are of course always perpendicular to the substrate. However the latter can be resolved using magnetic fields, thereby causing the particles to follow a helix path to the substrate. This is useful if one wants to coat a stent from the sides and not primarily on the abluminal sides. One practical application of a side-coating occurs when one wishes to implant a drug depot in a vessel to elute therapeutic nanoparticles to downstream tissue, which might be particularly useful for long diffuse diseases or cancer tissue.

The degradable inorganic adhesion layer can be disposed over the inorganic nanoparticles by methods known in the art. The adhesion layer can include one or more layers. In some embodiments, the adhesion layer is continuous and essentially non-porous. The adhesion layer can be formed by a self-limiting deposition process. In a self-limiting deposition process, the growth of the layer stops after a certain point (e.g., because of thermodynamic conditions or the bonding nature of the molecules involved), even though sufficient quantities of deposition materials are still available. For example, U.S. Provisional Patent Application 61/228,264, entitled “Medical Devices Having an Inorganic Coating Layer Formed by Atomic Layer Deposition,” filed Jul. 24, 2009, which is hereby incorporated by reference, describes a particularly useful process of atomic layer deposition (ALD).

Using ALD, the adhesion layer can have more uniformity in thickness across different regions of the device and/or a higher degree of conformality. A conformal layer is possible even for surfaces having very high aspect ratio structures (such as deep and narrow trenches, as would be the case for nanoparticles) and for surfaces within a porous system, wherein the surface is accessible from the outside via an interconnected network (e.g., interconnected nano- or micro-sized porosity). As used herein, “conformal” means that the coating follows the contours of the medical device geometry and continuously covers substantially all the surfaces of interest.

When depositing a plurality of aggregated particles on a surface, any number of techniques known in the art can be used to embed an inorganic nanoparticle into a carrier agent particle, e.g., a therapeutic agent particle. For example, a surface can be provided. Therapeutic agent particles, such as paclitaxel particles, can be provided using any method known to those of skill in the art. Of particular usefulness is Elan Drug Technologies's NanoCrystal® Technology—a commercial process that involves reducing the size of drug particles, typically to less than 2,000 nanometres. By reducing particle size, the drug's exposed surface area is increased. For example, using Elan's method, paclitaxel particles of about 100 nm in size can be provided. The plurality of therapeutic agent particles can be deposited on the surface, e.g., by spraying, inkjet deposition, roll coating, electrostatic spraying, and/or micropen coating. Then, a plurality of inorganic nanoparticles can be accelerated toward the plurality of deposited therapeutic agent particles using nanocluster deposition, to thereby deposit a quasi-soft landed layer of inorganic nanoparticles. As discussed above, particle sizing can be determined accurately using a quadrupole filter before deposition of the particles on the target. Mantis Deposition Ltd's Nanosys 500 combined with the MesoQ quadrupole mass filter can be used in-line with the NanoGen 50 system. For example, using the system, bias voltage can be set at 150 Volts with a deposition rate of 0.75 nm/minute (skilled practitioners will appreciate that such parameters will depend on the material used and nanoparticle size, among other parameters). The effect of the deposition voltage being slightly above soft-landing is that the inorganic nanoparticles become partially embedded in the therapeutic agent particles, thereby providing a plurality of aggregated particles. Alternatively or in addition, the method can be adapted to create aggregated particles in a system not associated with the surface of interest, followed by deposition of the aggregated particles onto the surface of interest. For example, therapeutic agent particles can be disposed initially on a surface and then coated using the Mantis system. The aggregated particles can then be washed or scraped off the surface and put into suspension and then coated onto the surface of interest using spray coating or other coating method.

In some embodiments, it may be desirable to roughen a surface of interest before performing depositions described herein. For example, a surface may be roughened to provide a series of nooks or invaginations on/within the surface. Any surface may be roughened, e.g., a metallic, polymeric or ceramic surface. Surfaces can be roughened using any technique known in the art. Particularly useful methods for roughening surfaces, such as the surfaces of a stent, are described, e.g., in U.S. Ser. No. 12/205,004, which is hereby incorporated by reference. The surface of a balloon may also be roughened. For example, a balloon surface may be roughened using a polarized laser beam (193 nanometer) using an energy density just below the ablation threshold. For example, for a PEBAX balloon the ablation threshold can be about 60 mJ cm⁻². The balloon shown in FIG. 5 was performed at 30 mJ cm⁻². The texture of the surface of the balloon that results after such treatment can serve as a protective “fur” for deposited particles (see, e.g., the texture of a balloon surface shown in FIG. 5). Using nanocluster deposition as described above to deposit a quasi-soft landed layer of inorganic nanoparticles onto such a balloon surface allows penetration of the nooks created by roughening of the surface. Expansion of the balloon in a passageway, e.g., a vessel, results in stretching and straightening of the surface, which causes the deposited material to be released from the nooks into the passageway or a tissue lining the passageway.

Further, as will be appreciated by skilled practitioners, particles described herein can be deposited on an entire surface of a device or onto only part of a surface. This can be accomplished using masks to shield the portions on which particles are not to be deposited. Further, with regard to stents, it may be desirable to deposit only on the abluminal surface of the stent. This construction may be accomplished by, e.g. coating the stent before forming the fenestrations. In other embodiments, it may be desirable to deposit only on abluminal and cutface surfaces of the stent. This construction may be accomplished by, e.g., depositing on a stent containing a mandrel, which shields the luminal surfaces.

The terms “therapeutic agent”, “pharmaceutically active agent”, “pharmaceutically active material”, “pharmaceutically active ingredient”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total. Generally, exemplary therapeutic agents include, e.g., sirolimus, everolimus, biolimus, zotarolimus, tacrolimus and paclitaxil.

Exemplary non-genetic therapeutic agents include anti-thrombogenic agents such heparin, heparin derivatives, prostaglandin (including micellar prostaglandin El), urokinase, and PPack (dextrophenylalanine proline arginine chloromethylketone); anti-proliferative agents such as enoxaparin and angiopeptin, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; antimicrobial agents such as triclosan, cephalosporins, aminoglycosides, nitrofurantoin, silver ions, compounds, or salts; biofilm synthesis inhibitors such as non-steroidal anti-inflammatory agents and chelating agents such as thylenediaminetetraacetic acid, O,O′-bis(2-aminoethyl)ethyleneglycol-N,N,N′,N′-tetraacetic acid and mixtures thereof; antibiotics such as gentamycin, rifampin, minocyclin, and ciprofloxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; βAR kinase (βARK) inhibitors; phospholamban inhibitors; proteinbound particle drugs such as ABRAXANE™; structural protein (e.g., collagen) cross-link breakers such as alagebrium (ALT-711); and/or any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (MCP-1) and bone morphogenic proteins (“BMPs”), such as, for example, BMP-2, BMP-3, BMP-4, BMP-5, BMP-6 (VGR-1), BMP-7 (OP-1), BMP-8, BMP-9, BMP-10, BMP-11, BMP-12, BMP-13, BMP-14, and BMP-15. Preferred BMPs are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNAs encoding them. Non-limiting examples of genes include survival genes that protect against cell death, such as antiapoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factors α and β, platelet-derived endothelial growth factor, platelet-derived growth factor, tumor necrosis factor a, hepatocyte growth factor, and insulin-like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase and combinations thereof and other agents useful for interfering with cell proliferation.

Exemplary small molecules include hormones, nucleotides, amino acids, sugars, and lipids and compounds having a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin−) cells including Lin−CD34−, Lin− CD34+, Lin−cKit+, mesenchymal stem cells including mesenchymal stem cells with 5-aza, cord blood cells, cardiac or other tissue-derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells. Any of the therapeutic agents may be combined to the extent such combination is biologically compatible.

Suitable medical devices include, but are not limited to, those that have a tubular or cylindrical like portion. A tubular portion of a medical device need not be completely cylindrical. The cross-section of the tubular portion can be any shape, such as rectangle, a triangle, etc., not just a circle. Such devices include, but are not limited to, stents, balloons of a balloon catheters, grafts, and valves (e.g., a percutaneous valve). A bifurcated stent is also included among the medical devices which can be fabricated by the methods described herein. The device can be made of any material, e.g., metallic, polymeric, and/or ceramic material.

The stents described herein can be configured for vascular, e.g. coronary and peripheral vasculature or non-vascular lumens. For example, they can be configured for use in the esophagus or the prostate. Other lumens include biliary lumens, hepatic lumens, pancreatic lumens, uretheral lumens and ureteral lumens.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6A1-4V, Ti-50Ta, Ti-lOIr), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

A stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., U.S. Pat. No. 6,290,721). The ceramics can be used with other endoprostheses or medical devices, such as catheters, guide wires, and filters.

### EXAMPLES

### Example 1

### Coated Stent

A Platinum Chromium ELEMENT stent (Boston Scientific) was cleaned using isopropanol and provided with a 50 nanometer thick layer of CeOx 5 nanometer diameter particles using the Mantis system Nanosys 500 (nanocluster deposition) combined with the The MesoQ quadrupole mass filter used in-line with the NanoGen-50. Bias voltage was set at about 200 to 500 Volt and deposition rate at 0.75 nm/minute. The sputter target was obtained from Goodfellow Corp: CE009100 cerium sputtering target purity 99.9%. The quadruople has an ultimate size resolution of better than 2% in filtering mode, allowing precise particle size definition to be achieved. The stents were mounted on a standard coating stent holder in an ALD machine. The stents were transported keeping them in their holders and placed in batch in the ALD machine, a Beneq TFS 500. The stents were preheated two (2) hours in TFS 500 to a temperature of 80° Celsius, after which a 2 nanometer aluminium oxide coating was deposited in 20 cycles using trymethylalumium (Sigma Aldrich 597775-5G, 99.9999% purity) and water as reactants. Finished stents were dismounted from the stent holders and crimped on balloons using standard processes.

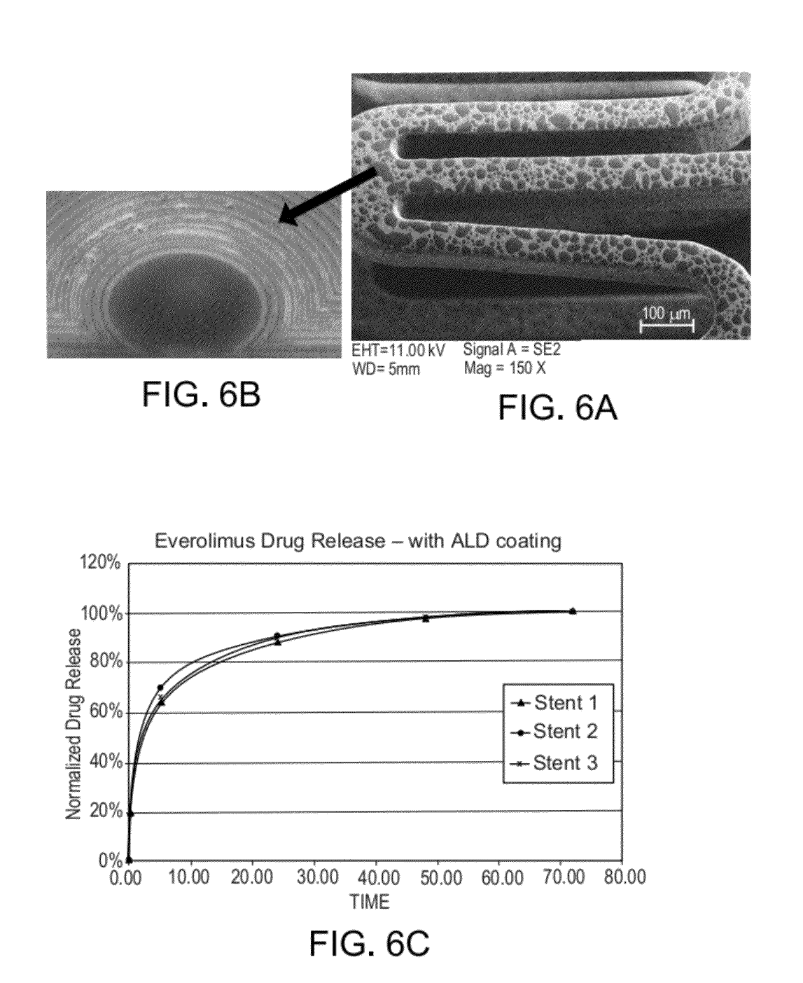

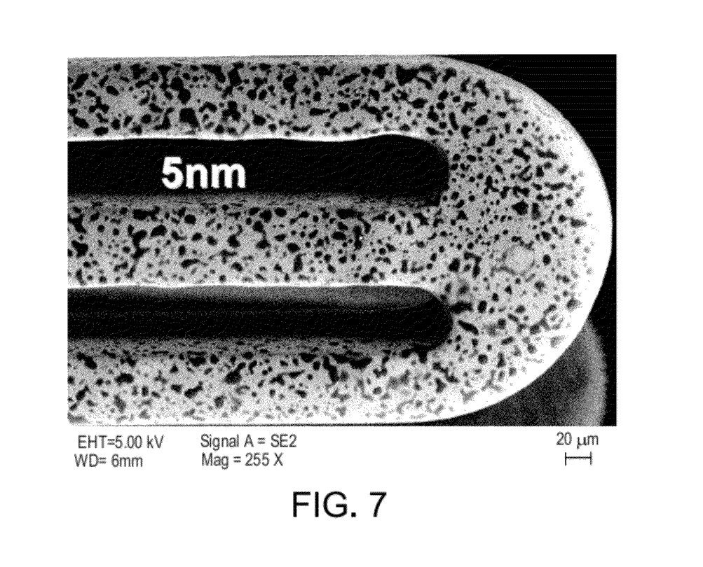

Using the output of this process, i.e., a stent coated with a layer of 5 nanometer CeOx nanoparticles conformally coated with a 2 nanometer dissolvable AlOx “glue” conformally coated on particles and stent, the stent was further provided with a layer of pure everolimus drug islands which were sprayed on top of the inorganic (cerium oxide+aluminum oxide) coating. The resulting configuration is shown in FIG. 6A, which is a close-up view of the resulting stent. For illustrative purposes, FIG. 6B is provided which shows a cross-section of a polystyrene microsphere coated with ALD, illustrating how ALD conformally coats particles. FIG. 7 is a different stent and is provided to show a stent at slightly higher magnification than that shown in FIG. 6B. The device was then replaced in the ALD machine where a second 18 nanometer thick layer of aluminium oxide coating was placed on top of drug layer and further surfaces. The process resulted in a device that released the drug up to 72 hours in a phosphate buffered saline medium spiked with Triton surfactant. FIG. 6C shows normalized drug release (0 to 1, 1 is 100% drug release) for several stents. This illustrates the results from an in vitro test that provides accelerated release. In vivo, this would represent release out to 30 days.

### Example 2

### Coated Balloon

Cerium oxide nanoparticles were produced in the following manner: A 0.07 g/mL (0.5 M) solution of hexamethylenetetramine (HMT) and a 0.016 g/mL (0.038 M) solution of Ce(NO3)3@6H2O were prepared and mixed separately for 30 min at room temperature. The nanoparticles were separated by centrifuging for 0.5 h at 12000 rpm in a Sorvall SLA-1500 rotor. The cerium oxide nanoparticles were washed with deionized water and dried. A solution of the cerium oxide nanoparticles was made by mixing 0.05 grams in a 100 ml solution of sodium heparin 10,000 U/mL (mean molecular weight 15000 Dalton, Leo Pharma Inc.) in water using the SonicSyringe system from Sono-Tek (Sono-Tek Corporation, Milton, New York). This system is designed for de-agglomeration of nanoparticles that have a tendency to clump and are difficult to keep evenly dispersed in suspension. A PEBAX balloon with a “furry” surface was created similar to that shown in FIG. 5 (50 polarized pulses of @ 30 mJ cm-2, LPXpro excimer laser (Coherent, Inc.)). In order to prevent the balloon surface to act as Velcro™ upon folding the balloon surface, the balloon was only partially treated with the laser treatment, such that three longitudinal strokes were treated around the circumference of the balloon surface, each being roughly equal to 60 degrees of the circumference. The heparin\ceriumoxide solution was coated on the balloon surface using the SonicSyringe in conjunction with an ultrasonic mist-nozzle system (Sono-Tek Corporation), set to 0.1 ml/min. The balloon was inflated at 5 bar and kept at a distance of 20 mm from the nozzle. The balloon was coated along longitudinal pathways covering just the laser treated sections. The coated balloon was dried at 50° C. for 2 hours and folded with three wings such that the balloon was positioned in the folding machine having the laser treated strokes forming one side of the balloon wings.

The foregoing description and examples have been set forth merely to illustrate the disclosure and are not intended to be limiting. Each of the disclosed aspects and embodiments of the present disclosure may be considered individually or in combination with other aspects, embodiments, and variations of the disclosure. Modifications of the disclosed embodiments incorporating the spirit and substance of the disclosure may occur to persons skilled in the art and such modifications are within the scope of the present disclosure.

What is claimed is:

1. A medical device, comprising:
a surface;
a plurality of inorganic nanoparticles disposed on at least a portion of the surface; and
a degradable inorganic adhesion layer, wherein the layer is disposed over the plurality of inorganic nanoparticles and adheres the nanoparticles to the surface.

2. The medical device of claim 1, wherein the medical device is an implantable device.

3. The medical device of claim 1, wherein the plurality of inorganic nanoparticles includes metal oxide nanoparticles.

4. The medical device of claim 1, wherein the plurality of nanoparticles comprises cerium, yttrium, titanium, iron, aluminum, tantalum, or gold nanoparticles, or a mixture of any two or more thereof

5. The medical device of claim 1, wherein the degradable inorganic adhesion layer comprises aluminum oxide, silicon dioxide, or zinc oxide.

6. The medical device of claim 1, wherein the plurality of nanoparticles comprises ultrasmall superparamagnetic particles of iron oxide (USPIOs) and cerium oxide nanoparticles.

7. The medical device of claim 1, wherein the plurality of nanoparticles comprises hybrid nanoparticles that comprise an iron oxide core and an outer cerium oxide coating.

8. The medical device of claim 1, wherein the degradable inorganic adhesion layer is of a thickness in the range of about 1 angstrom to about 1 micrometer.

9. The medical device of claim 1, further comprising a therapeutic agent disposed on the inorganic adhesion layer.

10. A medical device, comprising:
a surface; and
a plurality of aggregated particles disposed on the surface, wherein each aggregated particle comprises a carrier agent particle portion and an inorganic nanoparticle portion.

11. The medical device of claim 10, wherein the medical device is an implantable device.

12. The medical device of claim 10, wherein the surface is roughened and comprises a plurality of invaginations.

13. The medical device of claim 12, wherein at least a portion of the plurality of aggregated nanoparticles are disposed within the invaginations.

14. The medical device of claim 10, wherein the plurality comprises aggregated nanoparticles wherein the carrier agent particle portion comprises paclitaxel.

15. The medical device of claim 10, wherein the plurality comprises aggregated nanoparticles wherein the inorganic nanoparticle portions comprise cerium, yttrium, titanium, iron, aluminum, tantalum, or gold, or a mixture of at least two thereof

16. The medical device of claim 10, wherein the plurality comprises aggregated nanoparticles wherein the inorganic nanoparticle portions include ultrasmall superparamagnetic particles of iron oxide (USPIOs) and cerium oxide nanoparticles.

17. The medical device of claim 10, wherein the plurality comprises aggregated nanoparticles wherein the inorganic nanoparticle portions are hybrid nanoparticles comprising an iron oxide core and an outer oxide coating.

18. The medical device of claim 10, wherein in the size ratio of carrier agent nanoparticle to inorganic nanoparticle in at least a portion of the plurality of aggregated nanoparticles is in a range of about 2:1 to about 60:1.

19. A method of making a medical device, comprising:
providing a substrate comprising a surface;
depositing a plurality of inorganic nanoparticles on at least a portion of the surface, wherein the nanoparticles are in loose communication with the surface; and
depositing a degradable inorganic adhesion layer over the plurality of inorganic nanoparticles, wherein the adhesion layer adheres the nanoparticles to the surface.

20. The method of claim 19, wherein the inorganic nanoparticles are deposited using nanocluster deposition.

21. The method of claim 19, wherein the degradable adhesion layer is deposited using atomic layer deposition.

22. The method of claim 19, further comprising depositing a therapeutic agent on the degradable adhesion layer.

23. A method of making a medical device, comprising:
providing a substrate comprising a surface;
depositing a plurality of carrier nanoparticles on the surface; and
accelerating a plurality of inorganic nanoparticles toward the plurality of carrier agent nanoparticles, to thereby aggregate at least a portion of the plurality of inorganic nanoparticles to at least a portion of the plurality of carrier agent nanoparticles and provide a plurality of aggregated nanoparticles.

24. A method of making a medical device, comprising:
providing a substrate comprising a surface;
providing a plurality of aggregated nanoparticles, wherein the aggregated nanoparticles comprise a carrier agent nanoparticle portion and an inorganic nanoparticle portion; and
depositing the plurality of aggregated nanoparticles on the surface.

25. The method of claim 24, wherein the plurality of aggregated nanoparticles is provided by accelerating a plurality of inorganic nanoparticles toward a plurality of carrier agent nanoparticles, to thereby aggregate at least a portion of the plurality of inorganic nanoparticles to at least a portion of the plurality of carrier agent nanoparticles.

26. A method of making a medical device, comprising:
providing a substrate comprising a surface;
providing a plurality of hybrid nanoparticles, wherein the hybrid nanoparticles comprise a core that comprises iron oxide and an outer coating the comprises cerium oxide; and
depositing the plurality of hybrid nanoparticles on the surface.
